(19) United States
(12) Patent Application Publication
(10) Pub. No.: US 2019/0009072 A1
(43) Pub. Date: Jan. 10, 2019

(54) MALE AND FEMALE LUER CONNECTOR, AND LUER CONNECTION SYSTEM

(71) Applicants: Michael Schedler, Ramstein-Miesenbach (DE); Christopher Müller, Kaiserslautern (DE)

(72) Inventors: Michael Schedler, Ramstein-Miesenbach (DE); Christopher Müller, Kaiserslautern (DE)

(21) Appl. No.: 15/737,870

(22) PCT Filed: Jun. 17, 2016
(86) PCT No.: PCT/EP2016/064049

§ 371 Date: Dec. 19, 2017

(30) Foreign Application Priority Data
Jun. 19, 2015 (DE) 10 2015 211 370.1

(51) Int. Cl. A61M 39/10

(52) U.S. Cl. CPC A61M 39/1011; A61M 2039/1066; A61M 2039/1088; A61M 2039/1094; A61M 2039/1033

(57) ABSTRACT

Male Luer connector (200, 300) comprising a tubular body (210, 310) with a conical outer sheath (212) and a connection blocking element (230, 330) wherein the connection blocking element (230, 330) delimits a space around the tubular body (210, 310) and forms a codification stopper (240, 340) in axial direction (A). Female Luer connector (100) comprising a hollow reception body (110) with a conical reception (112) and a collar (120) at the outer end of the reception body (110) wherein the collar (120) comprises at least one codification notch (140). Connection system (400) comprising a male and a female Luer connector (100, 200, 300).

### 1. FIELD OF THE INVENTION

The present invention relates to male and female Luer and Luer-Lock connectors and a connection system comprising of one of a male and a female Luer or Luer-Lock connector, respectively, that exclude any mix-up between Luer connectors.

### 2. TECHNICAL BACKGROUND

Luer connection systems comprising male and female Luer connectors are known in the prior art in different embodiments. Luer connectors are standardized international with ISO standard 594 or Europe-wide with EN 20594 (or EN 1707) and Germany-wide with DIN EN 20594. The Luer connection system enables a manufacturing of manufacturer independent secure, leak-proof and sterile connections in particular in the medical-technical field. The standardized standard Luer connectors of different manufacturers can be combined arbitrarily with each other. For example, in using the Luer connection system syringes and canulae of different manufacturers can be combined arbitrarily with each other.

A male standard Luer connector comprises a tubular body that comprises a conical outer sheath. This conical outer sheath comprises a slope from the peak to the base of 6% according to the standard. The female standard Luer connector comprises a hollow reception body that comprises a conical hood-shaped reception that also comprises a slope of 6%. At the outer end of the female standard Luer connector it comprises a collar that often protrudes radially to the outside. The female standard Luer connector can be mounted onto the male standard Luer easily and can be connected secure and gas-proof by means of a friction fit. In a likewise easy manner the connection can be released again.

Besides the common standard Luer connection system also so called Luer-Lock connection systems are known in the prior art. They additionally comprise at the male Luer connector a thread that may engage into the radially protruding latching element in order to latch the male and female Luer connector by means of a rotation. By this, the Luer connection is guaranteeing tensile strength.

Commonly the female Luer connectors are arranged at a canula or a catheter or at the inlet of a conduit. Male Luer connectors are mostly arranged at a syringe, a feed tank, a medical device or at the outlet of a conduit.

The Luer connection systems known from the prior art have the disadvantage that due to the desired compatibility of the individual connectors among each other there are considerable risks of mix-ups. Thus, originally the Luer connection system was intended to be used according to the above-mentioned standards only for the connection between disposable syringes or injection canulae, respectively, and other medical devices, e.g. transfusion devices. Its easy and comfortable and secure handling, however, lead to a broad use also in different medical applications. Mix-ups that result from wrong connections can be dangerous to life for the patient.

Due to the fact that the Luer connection system is equally shaped for different applications like vascular and spinal or peridural, respectively, and partially also for enteral (e.g. feeding tubes) and respiratory applications, there is the risk that a medicine to be venously injected is wrongly applied spinal. For example, there is the risk that an intravenous port, at which a female Luer connector is arranged, is wrongly connected to a conduit with a male Luer connector, at which a medicine has to be applied enteral.

Such mix-ups and other problems have been discussed, among other things, in the technical report of DIN (DIN technical report 88:2000) from the year 2000 with the title “Luer connections—a report of the CEN Forum Task Group “Luer fittings”; German translation of the CR 13825:2000”.

From document WO 2008/009946 A1 a Luer connector system is known in which a male Luer connector is able to connect only to a particularly codified female Luer connector.

From document EP 1 083 963 A1 a special connection system comprising a security index for liquids in the medical field is known.

The technical problem to be solved by the present invention is, thus, to improve the Luer connection system and its male and female connector such that a mix-up of different medical systems is prevented during application in a cost-efficient way and without any extensive changes of the existing connection systems.

### 3. SUMMARY OF THE INVENTION

The above-mentioned problem is solved according to the invention by a male Luer connector according to claim 1, a female Luer connector according to claim 11 as well as a connection system comprising a male and a female Luer connector according to claim 15.

In particular, the above-mentioned problem is solved by a male Luer connector comprising a tubular body with a conical outer sheath, and a connection blocking element wherein the connection blocking element delimits a space around the tubular body and forms a codification stopper in axial direction.

The connection blocking element of the male Luer connector is configured, by means of its codification stopper arranged in axial direction, to mechanically prevent a connection of the male Luer connector with a female standard Luer connector or a standard Luer-Lock connector, but to enable a connection with a fitting female Luer connector that is inversely shaped. The codification stopper of the male connector is shaped complementary to a codification notch of the fitting female Luer connector such that these codification elements enable or prevent a connection of Luer connectors of the present invention according to the “key-lock-principle”. During connection of fitting Luer connectors the codification stopper is able to pass by the codification notch. The key-lock-principle of the Luer connectors of the present invention has the advantage that any mix-ups of Luer connectors, that are for example used at a patient at the same time, are excluded. In particular, a male Luer connector according to the invention prevents, for example for a medicine to be applied enteral, that this is wrongly connected to an intravenous port with a female standard Luer connector. The codification stopper, hereby, prevents a mounting of a female standard Luer connector.

Particularly, Luer connection systems that are used for a distinctive application field have a particular codification. A corresponding codification stopper, thus, comprises a defined geometric shape. Thereby, it is ensured that mix-ups of the Luer connection systems with each other are impossible because only these female Luer connectors can be connected to the codified male Luer connector that comprise a corresponding shape that fits to the codification stopper of the male Luer connector.

The connection blocking element of the male Luer connector can be injection molded preferably together with the tubular body such that existing production processes for these connectors can be maintained.

Due to the fact that the connection blocking element only delimits a space around the tubular body and forms a codification stopper the tubular body can be realized unchanged with its conical outer sheath according to the Luer standard. Thus, no problems will arise with regard to the leak-tightness of the connection or similar issues.

Preferably, the connection blocking element comprises at least one protrusion rigidly connected to the tubular body that extends axially in a tubular shaped space around the tubular body. The prevention of the connection by means of one or multiple protrusions is particularly advantageous because, hereby, a coupling of unfitting male and female Luer connectors is mechanically prevented in an easy way. The female standard Luer connector abuts during trying to connect to the protrusion of the male Luer connector and cannot mechanically surmount it. Furthermore, such protrusions that extend axially in a tubular shaped space around the tubular body can be manufactured easily, for example injection molded, and need only little additional material.

Preferably, the male Luer connector is a male Luer-Lock connector and further comprises a threaded body with an internal thread, wherein the codification stopper of the connection blocking element is attached to the outer end of the threaded body. Also at a male Luer connector with an internal thread the connection blocking element prevents with its codification faces the mechanical connection with an unfitting female Luer-Lock connector or a standard Luer-Lock connector.

Preferably, the connection blocking element is a separately manufactured, preferably hood-shaped, member that is connected to the male Luer connector. Thereby, the connection blocking element can be refitted easily to existing apparatuses or devices with a Luer connection. For this, the separately manufactured connection blocking element is attached to the existing male Luer connector, for example by mounting, adhering, screwing, welding or by similar attachment means. If the connection blocking element is preferably a hood-shaped member it can be for example mounted easily to a hood around the actual male Luer cone. For securing the connection it can additionally be adhered or welded. The hood can at the same time be the threaded body of a male Luer-Lock connector.

Preferably, the connection blocking element comprises two opposed codification stoppers. Thereby, a mechanical connection can be codified particularly advantageous that also cannot be connected wrongly in trying a diagonal connection. Thus, the likelihood of any mix-ups can be reduced further.

Preferably, the two opposed codification stoppers are shaped by two opposed protrusions.

Preferably, the codification stoppers are equally shaped. Thereby, the Luer connection can be connected in two different axial angle positions of the connectors towards each other.

Alternatively, the codification stoppers are differently shaped. Thereby, more codification possibilities are available in order to codify more different connection systems for different applications.

Preferably, the codification stopper, seen in axial direction, comprises a straight, triangular, rectangular, segment of a circle shaped or trapezoid silhouette. These shapes of the codification stopper result also in the present small dimensions of the Luer connectors in a secure codification.

Preferably, the connection blocking element is manufactured separately from the tubular body and comprises a hollow cylindrical reception with which the connection blocking element is connectable to the threaded body of the male Luer-Lock connector. This has the advantage that the connection blocking element can be manufactured more easily separate than together with the threaded body of the Luer-Lock connector. This leads to savings in the production of such male Luer-Lock connectors.

Alternatively, the connection blocking element is integrally formed with the male Luer connector. This is in particular advantageous since the integral formation of the male Luer connector according to the invention is particularly robust and a release of the connection blocking element from the male Luer connector is impossible. Apart from that, any installation procedures are omitted by an integral formation.

The above-mentioned problem is also solved by a female Luer connector, comprising a hollow reception body with a conical reception, and a collar at the outer end of the reception body, wherein the collar comprises at least one codification notch.

The codification notches of the female Luer connector are configured in such a way that the female Luer connector can be connected to an above described hereto fitting male Luer connector. Thereby, it is particularly advantageous that the female Luer connector can also be connected to a male standard Luer connector. However, due to the codification of the female Luer connector according to the invention by means of the codification notch it is possible to prevent dangerous mix-ups of the Luer connections.

Preferably, the female Luer connector is a female Luer-Lock connector and further comprises one or multiple radially protruding latching elements that are arranged at the collar of the female Luer connector. The one or multiple latching elements are configured in such a way that they may form a form fit with a thread of a male Luer-Lock connector and in particular may rotatably latch with the thread of the male Luer-Lock connector.

Preferably, the codification notch, seen in axial direction, comprises a straight, triangular, rectangular, segment of a circle shaped or trapezoid silhouette. The codification notches of the female Luer connector are preferably inversely shaped to the codification stoppers of the above described male Luer connector. The codification notches are, thus, shaped such that they comprise an inverse or corresponding, respectively, silhouette to the codification parts of the male Luer connector.

Preferably, the collar of the female Luer connector comprises two radial opposingly arranged codification notches. Thereby, a secure codification is ensured such that even in the case of a diagonal mounting of the female Luer connector an unfitting female Luer connector cannot be connected to the male Luer connector.

In a further preferred embodiment the collar of the female Luer connector comprises two asymmetrically arranged codification notches. For particular applications the codification notches can also be arranged asymmetrical or not mirror-inverted, respectively, for example in a 90° angle with respect to each other, at the collar of the female Luer connector. Thereby, no risk of rotation is given for example for a connection between surgery instruments. A defined axial angle position during connection of the female and male Luer connector may also be obtained in the preferred application of only one codification notch or in case of multiple differently shaped codification notches.

The above mentioned problem is also solved by a connection system comprising a male Luer connector as described above and a female Luer connector as described above, wherein the one codification notch or the multiple codification notches of the female Luer connector are inversely shaped to the one codification stopper or the multiple codification stoppers of the male Luer connector.

### 4. DESCRIPTION OF THE DRAWINGS

In the following, the present invention is described in more detail by reference to the accompanying figures, in which shows:

FIG. 1A to 1D an exemplary embodiment of a female Luer connector according to the invention in four different perspectives;

FIG. 2A to 2C an exemplary embodiment of a Luer connection system according to the invention comprising a male and a female Luer connector in the connected state in multiple perspectives;

FIGS. 3A and 3B an exemplary embodiment of a Luer-Lock connection system according to the invention comprising a male and a female Luer-Lock connector;

FIG. 4A to 4D the exemplary embodiment of the Luer-Lock connection system according to the invention of FIG. 3 in the connected state in multiple perspectives;

FIG. 5 a further exemplary embodiment of a Luer connection system according to the invention comprising a male and a female Luer-Lock connector;

FIG. 6A to 6C the exemplary embodiment of the Luer connection system according to the invention of FIG. 5 in the connected state in multiple perspectives;

FIG. 7 a further exemplary embodiment of a Luer connection system according to the invention comprising a male and a female Luer connector;

FIG. 8A to 8C the exemplary embodiment of the Luer connection system according to the invention of FIG. 7 in the connected state in multiple perspectives;

FIG. 9 a further exemplary embodiment of the Luer-Lock connection system according to the invention comprising a male and a female Luer-Lock connector;

FIG. 10A to 10D the exemplary embodiment of the Luer-Lock connection system according to the invention of FIG. 9 in the connected state in multiple perspectives;

FIG. 11A to 11D axial top views to exemplary embodiments of the Luer-Lock connection system according to the invention with different silhouettes of the codification stoppers and the codification notches;

FIG. 12 a further exemplary embodiment of a Luer connection system according to the invention comprising a male and a female Luer connector; and

FIG. 13A to 13D the exemplary embodiment of the Luer connection system according to the invention of FIG. 12 in the connected state in multiple perspectives.

### 5. DESCRIPTION OF PREFERRED EXEMPLARY EMBODIMENTS

In the following, by reference to the figures, preferred exemplary embodiments of the present invention are described:

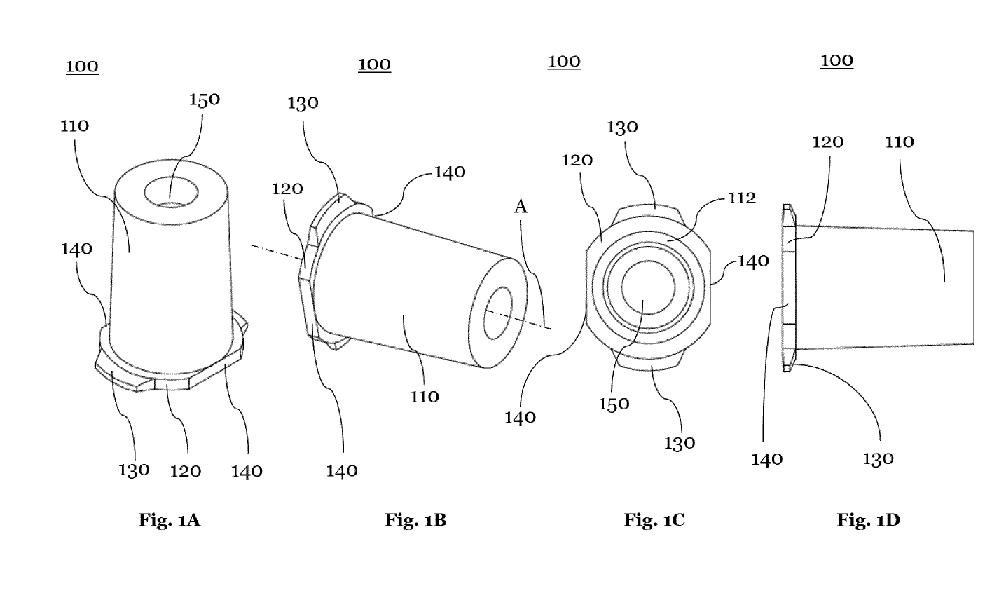

FIGS. 1A to 1D show an exemplary embodiment of a female Luer connector 100 in four different perspectives. The female Luer connector 100 comprises a hollow reception body 110 that includes a conical reception 112 on its inner side. The conical reception 112 comprises a slope of 6% typical for Luer connectors and serves the reception and sealing with a corresponding male Luer connector. The female Luer connector 100 further comprises a collar 120 that is arranged at the open end of the conical reception body 110. The collar 120 is preferably essentially in the shape of a circular ring with a rectangular cross section. Thereby, the female Luer connector 100 is basically compatible also to male standard Luer connectors (not shown) and may be connected to them.

Furthermore, the collar 120 comprises two opposing female codification notches 140 that comprise axial in the embodiment of FIGS. 1A to 1D, i.e. seen in connection direction, a straight edge and, thus, a straight silhouette, as can be seen in the axial view from below of FIG. 1C. The codification notches 140 are, thus, formed by two axial opposing straight parts of the collar 120.

Furthermore, the conical reception body 110 comprises two radially protruding latching elements 130. These are preferably arranged axially opposing at the collar 120 of the female Luer connector 100 and are conical reduced or slanted to the outside. The latching elements 130 serve to engage into a thread 322 of a male Luer-Lock connector 300 and to prevent any unwanted release with it by a rotation. In case of a Luer connection system 400 comprising a male Luer connector 200 without a thread 322 that is also called a male Luer-Slip connector the latching elements 130 can be omitted.

The female Luer connector 100 comprises furthermore an aperture 150 at the back end of the conical reception body 110 at which for example needles or conduits (not shown) may be connected.

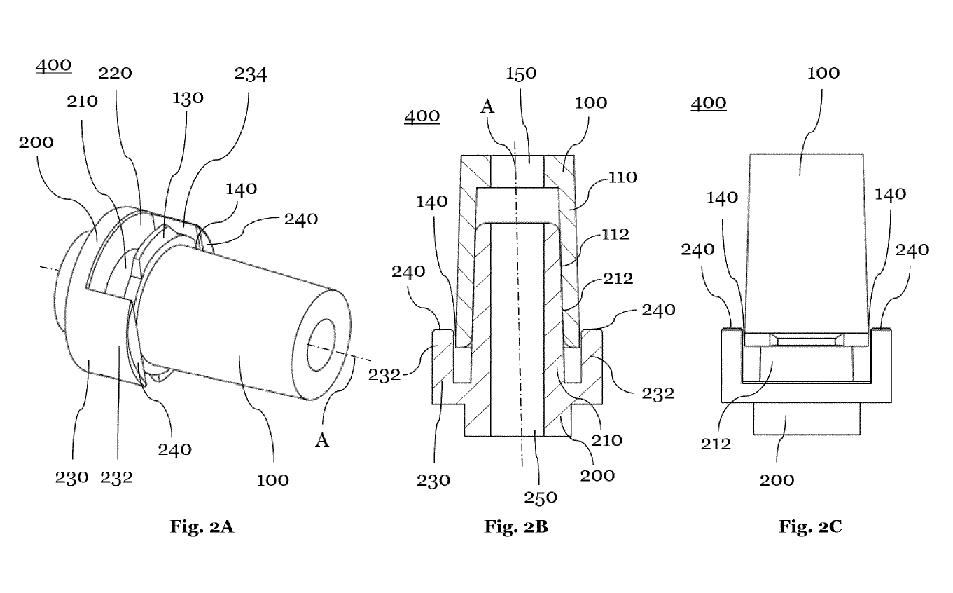

The FIGS. 2A to 2C show an exemplary embodiment of a Luer connection system 400 comprising a male Luer connector 200 (here a Luer-Slip connector 200) and a thereto fitting codified female Luer connector 100 in the connected state. The male Luer connector 200 comprises a tubular body 210. This tubular body comprises a conical outer sheath 212 which comprises a slope of 6% typical for a Luer connector and that serves for a connection and sealing with a corresponding female Luer connector 100. Furthermore, the tubular body 210 comprises a pass through aperture 250 that extends throughout the whole tubular body 210, as can be seen in FIG. 2B, and that provides a conduit for the flow of liquids or gases.

Furthermore, the male Luer connector 200 comprises a connection blocking element 230 that is formed in the exemplary embodiment of FIGS. 2A to 2C as two opposing protrusions 232. These protrusions 232 are rigidly connected to the tubular body 210 and extend radially in a space around the conical tubular body 210 and delimit this space. In particular, the two protrusions 232 form two codification stoppers 240 in axial direction. These codification stoppers 240 are geometrically codified by their silhouette in a direction to the tubular body 210 and thereby allow the axial mounting of a corresponding geometrically codified female Luer connector 100. Female Luer connectors that are uncodified or differently codified do abut with their collar 120 to the codification stoppers 240 and, thus, cannot be connected with the male Luer connector 200. By this mechanical codification of male and female Luer connectors 100, 200, 300 mix-ups during the connection of medical devices and ports can be avoided.

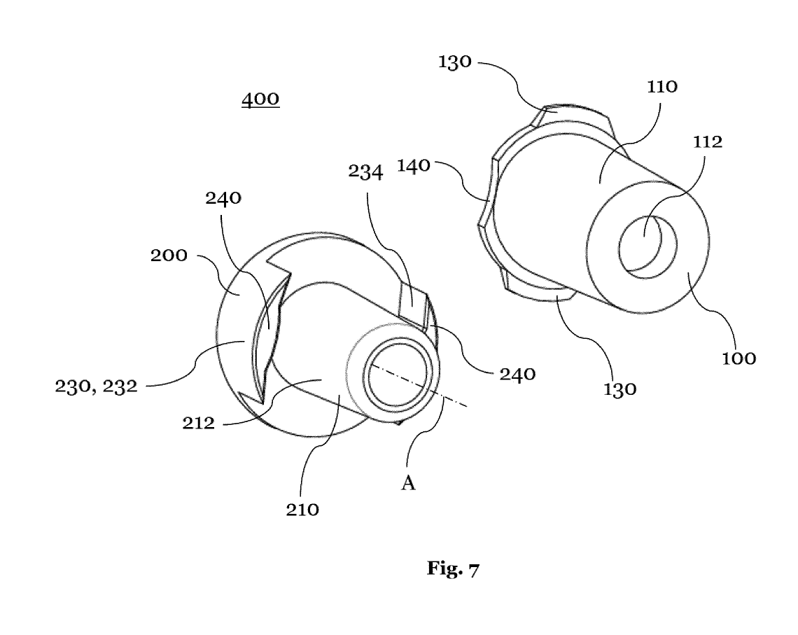

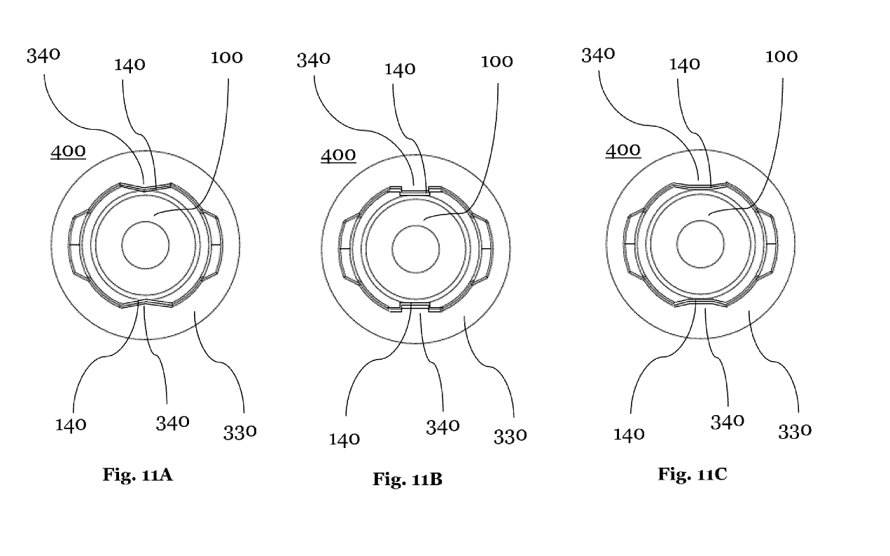

In the embodiment of FIGS. 2A to 2C the codification stoppers 240 comprise a straight edge, i.e. a straight silhouette in the direction to the tubular body 210 for codification. Correspondingly, thereto fitting female Luer connectors 100 also comprise a straight codification that is formed by a straight silhouette of the codification notch 140. A corresponding fitting female Luer connector is shown in FIGS. 1A to 1D. In FIGS. 7-9 Luer connectors with a silhouette of the codification stopper 240, 340 and the codification notch 140 in the shape of a section of a circle are shown. FIG. 11A shows a Luer connector comprising a triangular silhouette of the codification stopper 340 and of the codification notch 140. In FIG. 11B the silhouette of the codification stopper 340 and of the codification notch 140 is rectangular. In FIG. 11C the silhouette is trapezoid. The illustrated pairs of codification stopper 240, 340 and codification notch 140 fitting to each other are equally shaped at opposing sides of the Luer connector. However, it is also possible that the pairs of codification stopper 240, 340 and codification notch 140 fitting to each other are differently formed on opposing sides of the Luer connector in order to increase the codification possibilities. Furthermore, it is possible that only one pair of codification stopper 240, 340 and codification notch 140 fitting to each other or more than two pairs, like three, four or five pairs of pairs of a codification stopper 240, 340 and a codification notch 140 fitting to each other are provided at one Luer connection system 400.

The male Luer connector 200 can be connected to the fitting codified female Luer connector 100 because the female codification notches 140 and the male codification stoppers 240 are inversely shaped to each other. During the connection, the codification notches 140 of the fitting female Luer connector 100 are passed by on the inner surface 234 of the connection blocking element 230, as can be seen in FIGS. 2B and 2C, such that the connection blocking element 230 enables the connection. The connection stopper 240 is preferably formed as a plane perpendicular to the axial direction. The inner surface 234 of the connection blocking element 230 continues preferably parallel to the axial direction. The inner surface 234, however, can also be arranged in an angle to the axial direction, for example with an undercut, such that the space broadens behind the codification stopper 240.

A connection of the male Luer connector 200 with a female standard Luer connector is, thus, not possible, because the connection blocking element 230 mechanically prevents this connection. In such a case the female standard Luer connector abuts, during trying to connect, to the codification stoppers 240 of the connection blocking element 230 of the male Luer connector 200 and cannot be moved towards each other any further.

In the cut-view of FIG. 2 it can be seen that the conical outer sheath 212 of the tubular body 210 seals gas- and liquid-proof with the likewise conical reception 112 of the reception body 110. Thereby, the pass through aperture 250 of the male Luer connector 200 and the aperture 150 of the female Luer connector 100 form a channel for the exchange of liquid from the lower to the upper end of the Luer connection 400.

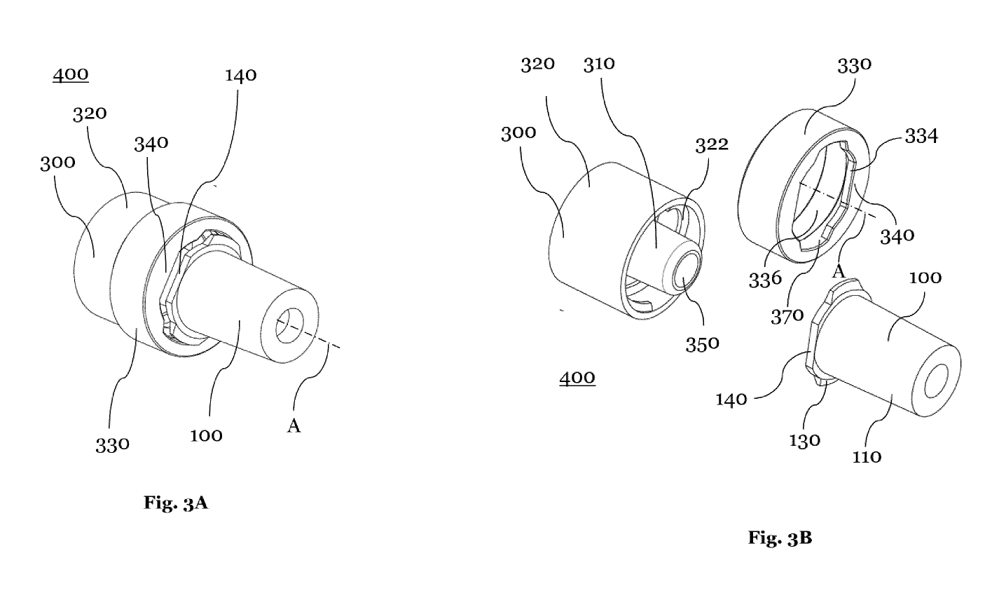

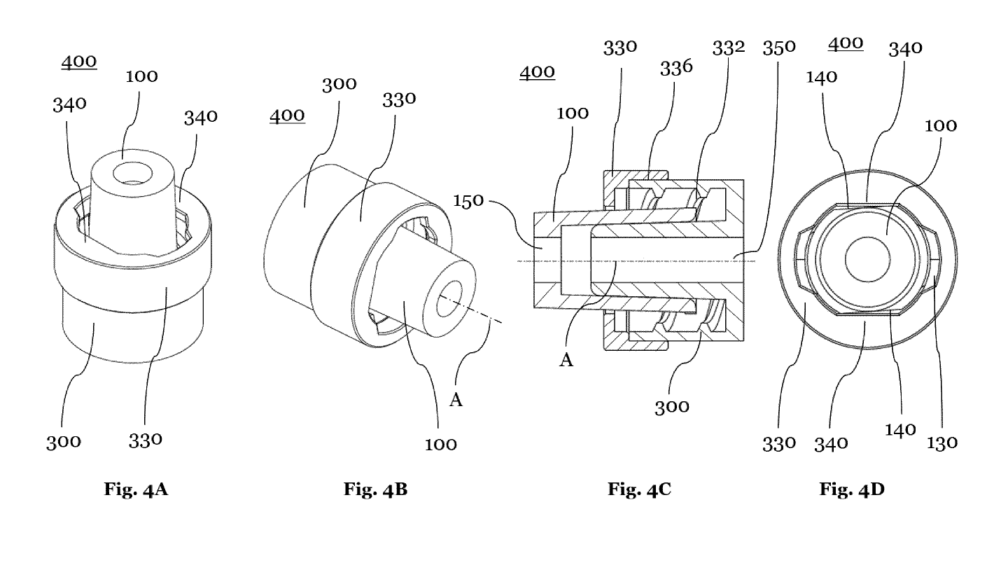

FIGS. 3A-4D show a further exemplary embodiment in the form of a Luer-Lock connection system 400 comprising a male 300 and a female Luer-Lock connector 100. FIG. 3A shows the connection system 400 in the state during the connection of the female Luer connector 100 with the male Luer-Lock connector 300.

FIG. 3B shows the exemplary embodiment of FIG. 3A as an exploded view with separate components. In this embodiment the tubular body 310 is integrally formed with the threaded body 320 that is formed hood-shaped and comprises an internal thread 322 on its inner side. The connection blocking element 330 is a separate hood-shaped member comprising a hollow cylindrical reception aperture 336 with which it can be arranged for securing to the threaded body 320 of the Luer-Lock connector 300. The connection blocking element 330 preferably includes two opposing codification stoppers 340 that are located after the installation at the outer end of the threaded body 320. The codification stoppers 340 codify by their shape of their axial visible silhouette the female Luer-Lock connectors 100 allowed for the connection. In the exemplary embodiment of FIGS. 3A to 4D the silhouette is straight such that the female Luer-Lock connector of FIGS. 1A to 1D also with a straight silhouette is fitting.

The attachment of the connection blocking element 330 on the threaded body 320 can be performed for example by adhering, welding or by friction fit. Then, the male Luer-Lock connector 300 includes the tubular body 310 with the threaded body 320 and the connection blocking element 330 comprising one or multiple codification stoppers 340.

For the establishment of the connection the female Luer connector 100 is passed through the connection blocking element 330 of the male Luer-Lock connector 300 and is in particular passed with its codification notches 140 on the inner surfaces 334 by the codification stoppers 340 of the codification blocking element 330, as can be seen in FIG. 3A. After that the female Luer-Lock connector is connected to the male Luer-Lock connector 300 by screwing wherein the radially protruding latching elements 130 engage behind the turns of thread 322. Thereby, both connectors 100, 300 are firmly connected with each other, as it is shown in FIGS. 4A to 4D.

The female Luer-Lock connector 100 can be connected to the male Luer-Lock connector 300, because the female codification notches 140 fit to the male codification stoppers 340 with respect to their axial silhouette and are in particular inversely shaped to each other. In case the female codification notches 140 do not fit to the male codification stoppers 340 the female Luer-Lock connector 100 abuts to the two codification stoppers 340 of the male Luer-Lock connector 300 and cannot be connected with it.

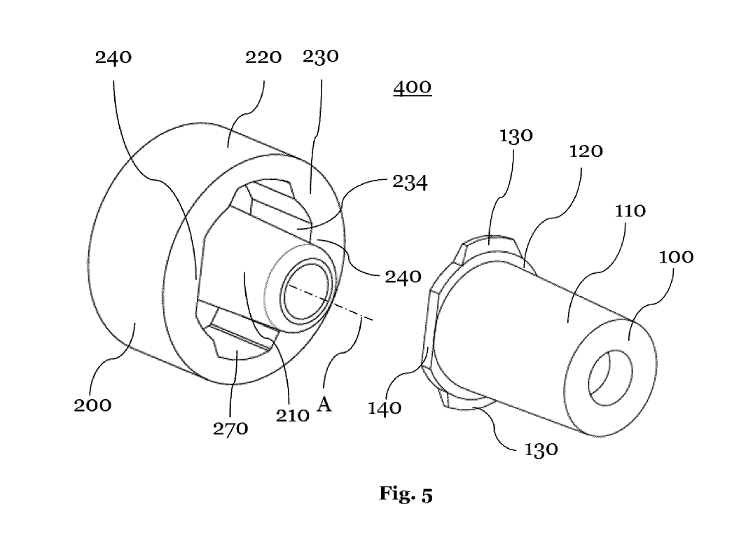

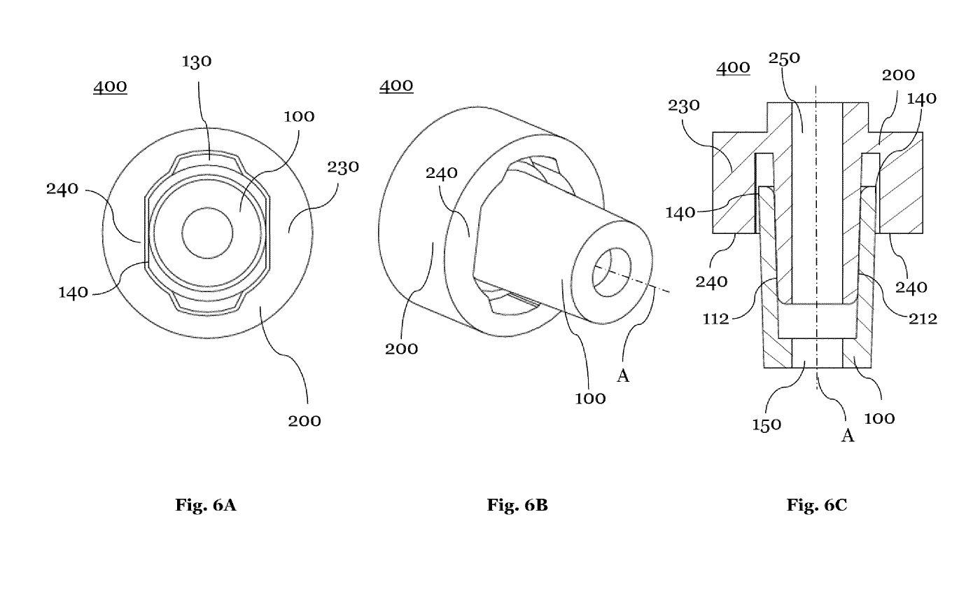

FIGS. 5 to 6C show a further exemplary embodiment of a Luer connection system 400 comprising a male Luer connector 200 and a female Luer connector 100. In this exemplary embodiment a hood-shaped connection blocking element 230 and the tubular body 210 of the male Luer connector 200 are integrally formed. This has the advantage of a simple manufacturing and a particularly robust construction. In contrast to FIG. 2A the conical tubular body 210 of the male Luer connector 200 is enclosed in this embodiment radially by the hood-shaped connection blocking element 230.

The connection is established correspondingly by an axial pushing of the tubular body 210 of the male Luer connector 200 into the reception body 110 of the female Luer connector 100.

This exemplary embodiment can also be realized as a Luer-Lock connection system 400 in which the male Luer connector, a male Luer-Lock connector 300, as described above for the male Luer connector 200, is correspondingly integrally formed with the connection blocking element 230.

FIGS. 6A to 6C show further views of the exemplary embodiment of FIG. 5 in the connected state. Thereby, FIG. 6A is a top view, FIG. 6B is a three-dimensional view and FIG. 6C is a cross-sectional side-view of the exemplary embodiment of FIG. 5 in the connected state. In FIG. 6A, in particular the invers corresponding shapes and silhouettes, respectively, of the female codification notches 140 and the male codification stoppers 240, respectively, can be seen.

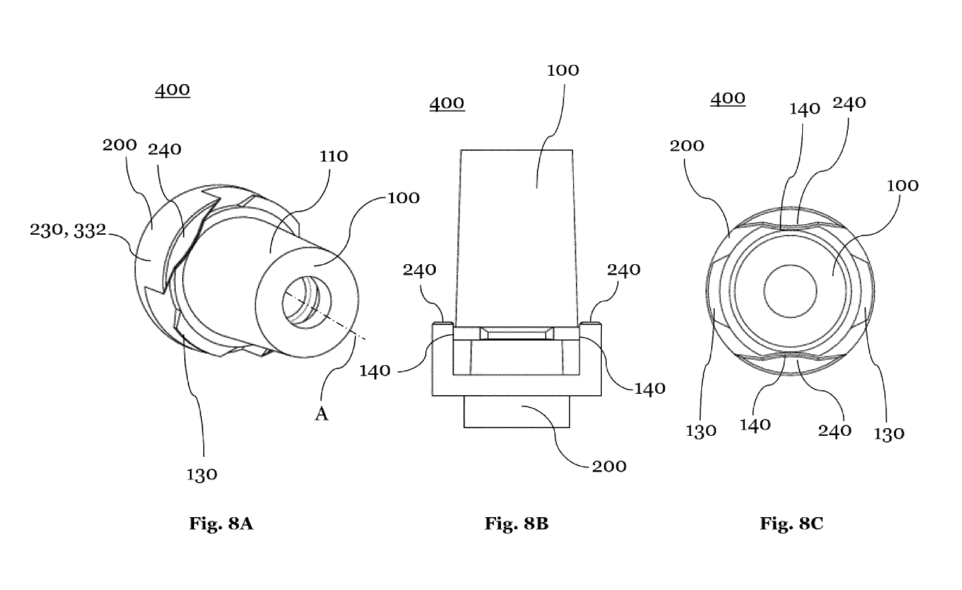

FIG. 7 shows a further exemplary embodiment of a Luer connection system 400. Hereby, the male codification stoppers 240 of the male Luer connector 200 and the female codification notches 140 of the female connector 100 are realized in the shape of a section of a circle, as can be seen particularly well in the top view of FIG. 8C.

FIG. 8A to 8C show further views of the exemplary embodiment of FIG. 7 in the connected state. Thereby, FIG. 8A is a three-dimensional view and FIG. 8B is a side-view of the exemplary embodiment of FIG. 7 in the connected state.

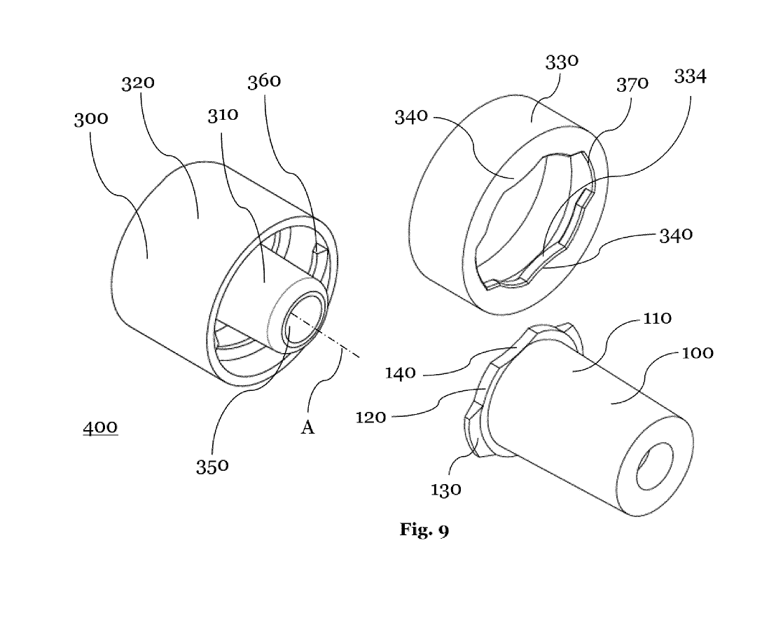

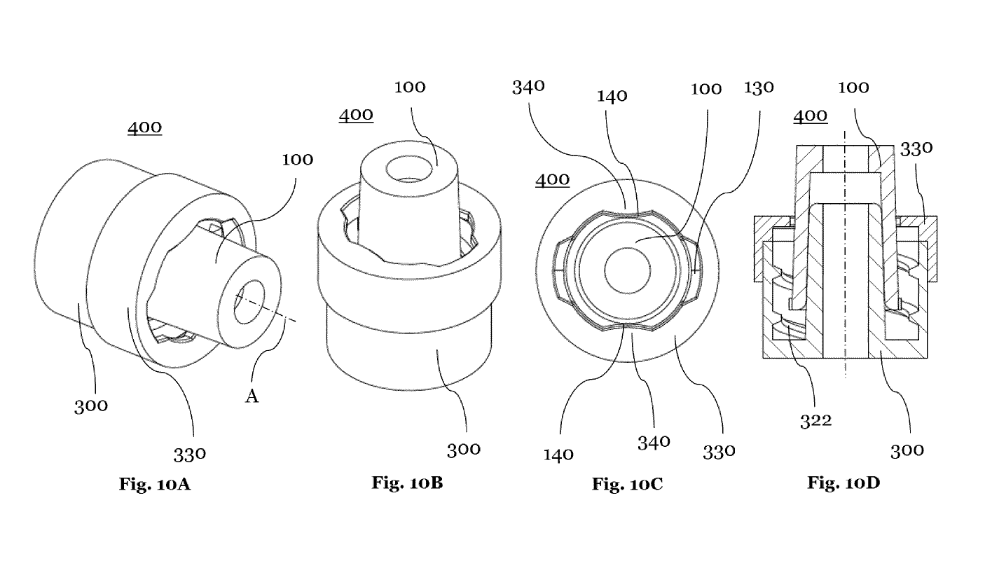

FIGS. 9 to 10D show a further exemplary embodiment of a Luer connection system comprising a female Luer connector 100, similar to FIG. 7, and a male Luer-Lock connector 300, similar to FIG. 3B, that includes a tubular body 310 with a threaded body 320 and a separate hood-shaped connection blocking element 330. The tubular body 310 comprises a pass through aperture 350 for passing through gases and liquids. The two codification stoppers 340 of the connection blocking element 330 are, here, formed in their axial silhouette in the shape of a section of a circle such that they fit with inversely formed codification notches 140 with the shape of a section of a circle of the female Luer connector 100.

Alternatively, the tubular body 310 and the connection blocking element 330 can be integrally formed similar to the exemplary embodiment of FIG. 5.

FIGS. 10A to 10D show views of the exemplary embodiment of FIG. 9 in the connected state. Thereby, the FIGS. 10A and 10B are three-dimensional views, FIG. 10C is a top view and FIG. 10D is a cross-sectional side-view of the exemplary embodiment of FIG. 9 in the connected state. In FIG. 10C, in particular the inversely corresponding shapes of the female codification notches 140 and the male codification stoppers 340, respectively, with the axial silhouette in the shape of a section of a circle can be seen well.

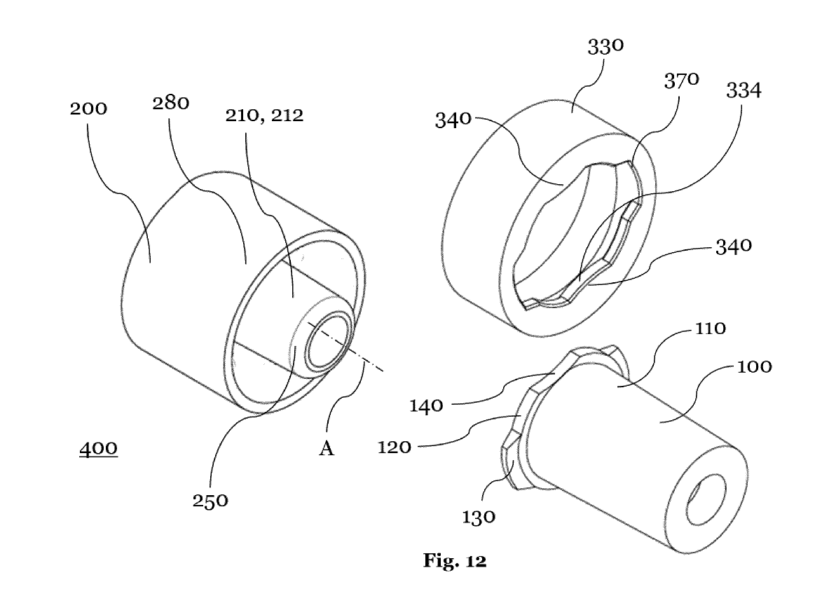

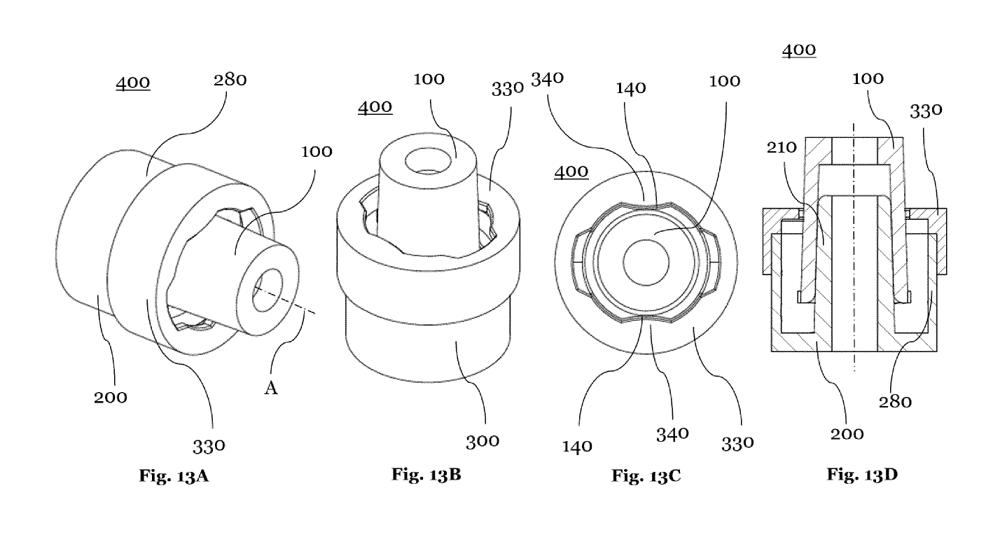

The FIGS. 12 to 13D show a further exemplary embodiment of a Luer connection system 400 comprising a female Luer connector 100, similar to FIG. 7 and FIG. 9, and a male Luer connector 200, similar to the male Luer-Lock connector of FIG. 3B, but without an internal thread. The illustrated Luer connector 200 is, thus, a Luer-Slip connector 200. This comprises around the actual tubular body 210 with conical outer sheath 212 a hood 280 onto which a separate hood-shaped connection blocking element 330 may be mounted. Thereby, the connection blocking element 330 may, as shown, preferably be the same as it is used for the Luer-Lock connection system of FIGS. 9-10D. Thus, the connection blocking element 330 is universal useable which, among other things, reduces manufacturing costs. The tubular body 210 comprises, as ever, a pass through aperture 250 for passing through gases and liquids. The hood 280 can preferably be integrally formed, as can be seen in FIG. 13D, with the tubular body 210 of the male Luer connector 200. The two codification stoppers 340 of the connection blocking element 330 are realized here in its axial silhouette in the shape of a section of a circle such that they fit with inversely formed codification notches 140 in the shape of a section of a circle of the female Luer connector 100.

Alternatively, the tubular body 210 and the connection blocking element 330 can be integrally formed, similar to the embodiment of FIG. 5 that is, however, more difficult to realize in the production than the one shown solution with multiple parts with a separate connection blocking element 330.

FIGS. 13A to 13D show views of the exemplary embodiment of FIG. 12 in the connected state. Thereby, the FIGS. 13A and 13B are three-dimensional views, FIG. 13C is a top view and FIG. 13D is a cross-sectional side-view of the exemplary embodiment of FIG. 12 in the connected state. In FIG. 13C, in particular the inversely corresponding shapes of the female codification notches 140 and the male codification stoppers 340, respectively, with the axial silhouette in the shape of a section of a circle, can be seen well.

In all of the exemplary embodiments that relate to a Luer connection system 400 comprising a female Luer connector 100 comprising latching elements 130, the male Luer connector 200, 300 may comprise correspondingly one of the shapes of the latching element 130, as for example shown in FIG. 5, that includes an inverse recess 270, 370 for the latching elements 130.

Due to the different exemplary embodiments of the present invention a Luer connection system 400 is described exemplarily by which mix-ups between Luer connectors 100, 200, 300 can be effectively prevented and, thus, the safety of patients can be increased significantly in an easy and cost-effective manner.

1. A male Luer connector comprising:
a. a tubular body with a conical outer sheath; and
b. a connection blocking element;
c. wherein the connection blocking element delimits a space around the tubular body;
d. forms a codification stopper in axial direction; and
e. is configured to mechanically prevent a connection of the male Luer connector with a female standard Luer connector and a female standard Luer-Lock connector.

2. The male Luer connector according to claim 1, wherein the connection blocking element comprises at least one protrusion rigidly connected to the conical tubular body that extends axially in a tubular shaped space around the tubular body.

3. The male Luer connector according to claim 1, wherein the male Luer connector is a male Luer-Lock connector and further comprises a threaded body with an internal thread, wherein the codification stopper of the connection blocking element is attached to the outer end of the threaded body.

4. The male Luer connector according to claim 1, wherein the connection blocking element comprises two opposed codification stoppers.

5. The male Luer connector according to claim 4, wherein the two opposed codification stoppers are shaped by two opposed protrusions.

6. The male Luer connector according to claim 4, wherein the codification stoppers are equally shaped.

7. The male Luer connector according to claim 4, wherein the codification stoppers are differently shaped.

8. The male Luer connector according to claim 1, wherein the codification stopper, seen in axial direction, comprises a straight, triangular, rectangular, segment of a circle shaped or trapezoid silhouette.

9. The male Luer connector according to claim 3, wherein the connection blocking element is manufactured separately to the tubular body and comprises a hollow cylindrical reception with which the connection blocking element is connectable to the threaded body of the male Luer-Lock connector.

10. The male Luer connector according to claim 1, wherein the connection blocking element is integrally formed with the male Luer connector.

11. A female Luer connector, comprising
a. a hollow reception body with a conical reception, and
b. a collar at the outer end of the reception body, wherein
c. the collar comprises at least one codification notch.

12. The female Luer connector according to claim 11, wherein the female Luer connector is a female Luer-Lock connector and further comprises one or multiple radially protruding latching elements that are arranged at the collar of the female Luer connector.

13. The female Luer-Lock connector according to claim 11, wherein the codification notch, seen in axial direction, comprises a straight, triangular, rectangular, segment of a circle shaped or trapezoid silhouette.

14. The female Luer connector according to claim 11, wherein the collar comprises either
a. two radial opposingly arranged codification notches; or
b. two asymmetrically arranged codification notches.

15. A connection system comprising a male Luer connector according to claim 1 and a female Luer connector comprising
a. a hollow reception body with a conical reception, and
b. a collar at the out end of the reception body, wherein
c. the collar comprises at least one codification notch, wherein the one codification notch or the multiple codification notches of the female Luer connector are inversely shaped to the one codification stopper or the multiple codification stoppers of the male Luer connector.
